(19) United States
(12) Patent Application Publication
(10) Pub. No.: US 2025/0332027 A1
(43) Pub. Date: Oct. 30, 2025

(54) SYRINGE FOR INTRAOCULAR INJECTION

(71) Applicant: Juan Bautista Yepez Martinez, Maracaibo (VE)

(72) Inventor: Juan Bautista Yepez Martinez, Maracaibo (VE)

(21) Appl. No.: 18/645,488

(22) Filed: Apr. 25, 2024

(51) Int. Cl. A61F 9/00

(52) U.S. Cl. CPC A61F 9/0008

(57) ABSTRACT

A syringe for intraocular injection that allows injection procedures to be conducted in the eye of a patient with benefits related to ergonomics and more comfortable postures for the operator that allow a safer and more reliable application by compressing a portion of a casing with the fingers containing a medical solution.

### TECHNICAL FIELD

The present invention belongs to the field of medicine, specifically to the field of devices specially configured for the administration of a liquid solution in some part of a patient's body, more specifically in the eye of a patient.

### BACKGROUND OF THE INVENTION

It is known that intraocular injections consist of the administration of medications in the eye to treat ocular diseases and protect vision where the medication is injected to treat some diseases caused, for example, by degenerative problems, choroidal neovascular membrane problems that secondary to several diseases (for example, high myopia or central serous choroidopathy), macular edema (thickening of the macula), as well as infections or inflammations inside the eye and are necessary because the effects of the administration of a medication orally or by vein would not reach the interior of the eye with the adequate intensity to be able to carry out the therapeutic activity with maximum efficiency. Furthermore, in some cases, to reach the necessary levels, the doses that must be taken orally will be too high and could even be harmful to other organs in the body.

Normally the type of medications injected are antibiotics such as anti-VEGF drugs (faricimab [Vabysmo], aflibercept [Eylea®], bevacizumab [Avastin®], ranibizumab [Lucentis®]) or corticosteroids (dexamethasone [Ozurdex]).

Intraocular injections are typically performed in the operating room using topical anesthesia. Before proceeding, the eye and eyelids are disinfected, and after the injection, patient may experience a foreign body sensation, pressure in the eye or mild, non-specific discomfort, although there is usually no pain. A small subconjunctival hemorrhage may also occur, although these effects will disappear over time.

It is also known that personnel who give these types of injections due to their unique job are more likely to suffer muscle imbalances during intraocular injections due to an uncomfortable hand position when holding a syringe because doing so complies with the risk conditions for presenting a musculoskeletal disorder (MSD), which are the following:
Repetition: the act of doing the same task with the same muscles using them repeatedly.
High Force: high muscle power in lifting and grip.
Uncomfortable posture: joints bent out of their normal position because when holding a conventional syringe, the thumb is away from the index and middle fingers.
Hand-arm vibration: coming from tools or equipment that involve movement.

In addition, it is known that retina specialists know very well that, for example, intravitreal injection can cause the presence of silicone droplets in the vitreous and that the droplets come from the syringes used for the injection because most are made with silicone oil to allow better advancement of the plunger. However, a less invasive cause occurs in the needles since almost all needles are immersed in a bath of silicone oil to cover the outer surface and allow better penetration into the tissue, which in any case is less harmful than silicone. to allow the displacement of the plunger.

Continuing with the example of intravitreal injection, it is known that this allows highly targeted pharmacological therapy, maximizing the administration of therapeutic drugs to the posterior pole and at the same time minimizing systemic toxicity. With the increasing use of intravitreal anti-VEGF agents in the treatment of neovascular age-related macular degeneration (AMD), diabetic macular edema, retinal vein occlusion, and various other retinal vascular disorders, injection Intravitreal surgery has become the most common ophthalmic procedure performed in the world.

Possible complications of intravitreal injections have been described, such as intraocular inflammation/infection, retinal detachment, traumatic lens damage, intraocular hemorrhage, and hyper/hypotonia. Challenges associated with these injections include stabilizing the eyeball before inserting the needle, moving a finger quickly and stably to apply leverage after inserting the needle, injecting precise amounts of small volumes of fluid by pressing the plunger with the same or other hand (or the hand of an assistant), and carefully removing the needle without damaging any structure.

The correct execution of the intravitreal injection is important to avoid the risks associated with the procedures. A successful injection depends on careful placement of the injection site and control of the needle during the injection. This requires the injection provider to administer a controlled injection while stabilizing the patient's eye, a task that sometimes requires two people.

Although there are patient factors that pose risks, there are also factors associated with the person giving the injection since the level of precision and accuracy depends on the provider's skill in the injection, placing this quality in a significant role. Given that the skills depend on each person for the application of an injection, the component involved in the application of the injection that can be worked on is the syringe, providing a structure that has a series of advantages to cause the level of skill of the syringe supplier requires in any case fewer elements to achieve an adequate injection, reducing the possibility of failure.

In addition, syringes for the application of intraocular injections include elements traditionally used in these devices such as:
1. Cylinder or body: it is the main and elongated part of the syringe where the medication is placed.
2. Plunger or piston: it is a thin and mobile piece that is located inside the cylinder and that allows the liquid to be pushed or extracted by means of pressure.
3. Needle: It is the thinnest and most pointed part of the syringe, which is inserted into the eye to inject a medication.
4. Needle cap: it is a protection that covers the needle before use to prevent contamination.
5. Needle protector: it is a plastic piece that is placed on the tip of the needle to protect it and prevent accidental contact.
6. Cone: it is a plastic piece found at the top of the syringe that allows the needle to be attached safely.
7. Scale: it is a graduation usually in milliliters that is found on the body of the syringe and allows you to measure the amount of liquid to be administered.
8. Locking ring: it is a piece found at the end of the cylinder that allows the plunger to be fixed or released in its position.

Although there are elements of the syringe that are essential for the application of an intraocular injection, such as the needle or the needle holder, and others that do not really affect the operation of the syringe, such as the graduation; and displacement elements such as the piston or variants thereof represent a risk during application since their manipulation requires the forced intervention of the thumb, index and middle fingers where the thumb is used to hold the upper part of the syringe and control the displacement of the plunger, while the index and middle fingers are used to hold the bottom of the syringe and guide the needle towards the eye, causing an uncomfortable and biomechanically inefficient posture due to the increase in the elements involved that eventually increase the risk of failure or error due to loss of control during injection combined with the fact that the sensitivity or touch with respect to the displaced liquid is relatively indirect because it is the plunger that pushes the fluid.

Another disadvantage is the level of waste derived from the application of an intraocular injection with a conventional syringe since the amount of plastic required to manufacture this device is considerable due to the number of parts to be assembled that, once discarded, become potential contaminants in case of inappropriate handling.

In view of the above, there is currently a need for a device for applying an injection to the eye of a patient in an intraocular injection procedure that is effective, safe, sensitive, relatively simple, and intuitive to operate, but at the same time that is dependable in terms of precision and accuracy. Additionally, there is a need to reduce the elements involved in an injection procedure associated with a syringe, such as the plunger, piston and other means of displacement that are normally made of plastic and thereby reduce the environmental impact and, therefore, provide a intraocular injection syringe that provides a simple, reliable injection application configuration and with greater control of the application through an ergonomic configuration for health personnel with the intention of reducing the risk of error or failure in an injection application event.

### SUMMARY OF THE INVENTION

It is therefore a main object of protection, a syringe for intraocular injection that comprises a sealed casing and isolated from the outside that houses a liquid drug where the casing comprises a first casing part joined to a second casing part where both are reservoirs. of the liquid drug and comprise an intermediate central passage means that allows the fluid to be present in both internal parts of the casing, where the first part of the casing has a more rigid configuration than the second part of the casing where the latter maintains a compressible configuration as well as a breakable termination where the base of a needle is connected to achieve the expulsion of the fluid, said compressible configuration being allowed by the reduction in the thickness of the walls of the second part of the casing with respect to the thickness of the walls. of the first part of the casing.

Another object of protection is a syringe for intraocular injection that can be intravitreal, intracameral, intracorneal and suprachoroidal injections of medications indicated in the treatment of different types of ocular diseases where this syringe can be conceived as a device for applying a solution that requires a minor intervention by medical personnel since they can apply an injection using three fingers (thumb, index and middle) in a more comfortable position (closer separation between fingers) in a kind of holding a pencil to exert pressure on a portion of the syringe and get the medicine out.

Another object of protection is a syringe for intraocular injection free of a traditional syringe configuration such as thumb rest, finger flange, plunger and rubber stopper that are potential contaminants once the syringe has been discarded.

Another object of protection is a syringe for intraocular injection that provides a prefilled liquid solution.

Another object of protection is a syringe for intraocular injection that has a rigid portion to protect the contents or prefilled liquid solution.

Another object of protection is a syringe for intravitreal injection that has a flexible, compressible, or squeezable portion to achieve the expulsion of a liquid into the eye of a patient.

Another object of protection is a syringe for intraocular injection that has coupling means for a needle.

Another object of protection is a syringe for intraocular injection that has protection means for an attached needle.

Another object of protection is a syringe for intraocular injection that can be managed with one hand.

Another object of protection is a syringe for intraocular injection containing reference means for establishing an application zone for an intravitreal injection.

Another object of protection is a syringe for intraocular injection that has identification means labeled, printed, or even belonging to the body of the syringe.

Another object of protection is a syringe for intraocular injection that has graduation means labeled, printed, or even belonging to the body of the syringe.

Another object of protection is a syringe for intraocular injection that is transparent and allows the prefilled content of a liquid solution to be visualized.

Another object of protection is a syringe for intraocular injection that is not transparent and does not allow the prefilled content of a liquid solution to be visualized.

Another object of protection is an environmentally friendly syringe for intraocular injection.

Another item of protection is an intraocular injection syringe that requires a lower amount of tension on the muscles and tendons of the hand to reduce the risk of a misapplied injection.

Another object of protection is a syringe for intraocular injection that provides better biomechanical positioning of the fingers of medical personnel for efficient operation.

Another object of protection is a syringe for intraocular injection to reduce the risk of musculoskeletal disorders (MSDs).

Another object of protection is a syringe for intraocular injection to decrease operator error and reduce adverse outcomes.

Another object of protection is an intraocular injection syringe that limits the potential for adverse outcomes and improves safety and control during intraocular injections.

A fundamental aspect of the syringe for intraocular injection lies in the ergonomic design that allows the syringe to be manipulated to achieve the release of fluid only by applying pressure in a compressible area through the intervention of the index finger, thumb and middle finger and even with some Use thumb and index fingers for better stabilization and control during injection.

The syringe for intraocular injection has several advantages such as:
Ergonomic design (rigid tube and piston not included).
Caliper included, (optional).
Shorter injection preparation time.
Manage only with one hand, assistant or second hand, not necessary.
Better position for easier injection.
Less risk of suffering from musculoskeletal disorders (MSD), less uncomfortable postures.
More environmentally friendly. (Less amount of plastic).

Even in a further aspect the same principle can be applied to diverse types of injection techniques, such as, peribulbar, intracameral, intrastromal, intradermal, intra-articular, intramuscular, and many others, simply by modifying the size of the container and needles.

### BRIEF DESCRIPTION OF THE FIGURES

FIG. 1 shows an isometric view of the syringe for intraocular injection;

FIG. 2 shows an isometric view of the syringe for intraocular injection;

FIG. 3 shows a side view of the syringe for intraocular injection;

FIG. 4 shows an isometric view of the syringe for intraocular injection with a middle section to visualize its internal configuration;

FIG. 5 shows a side view of the syringe for intraocular injection to visualize its internal configuration;

FIG. 5A shows a sectional side view of the syringe for intraocular injection to visualize its internal configuration;

FIG. 6 shows an isometric view of the syringe for intraocular injection with a compression on its compressible portion;

FIG. 7 shows a side view of the syringe for intraocular injection with a compression in its compressible portion;

FIG. 8 shows a side view of the syringe for intraocular injection with a compression in its compressible portion;

FIG. 9A shows a side view of the syringe for intraocular injection with some labeling modes;

FIG. 9B shows another side view of the syringe for intraocular injection with some labeling modes;

FIG. 9C shows another side view of the syringe for intraocular injection with some labeling modes; and

FIG. 10 shows a side view of the syringe for intraocular injection with a labeling mode.

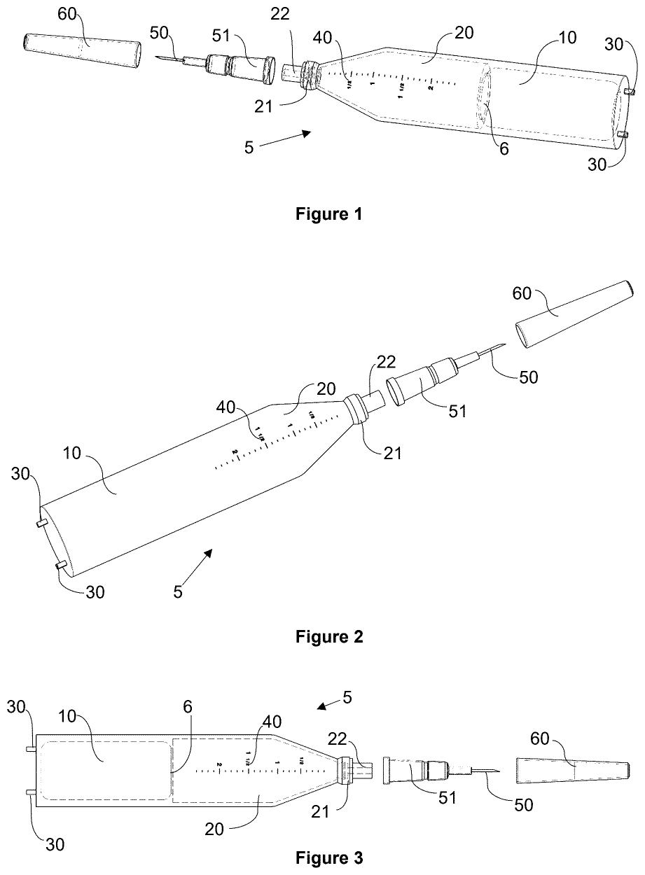

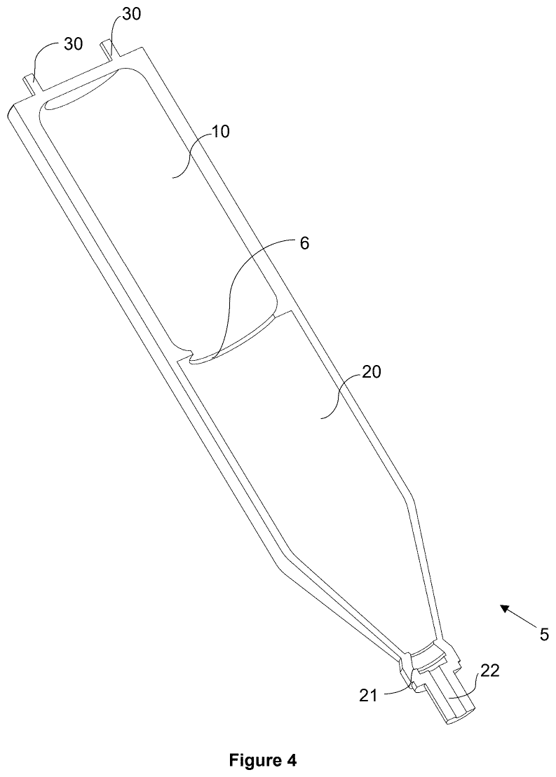

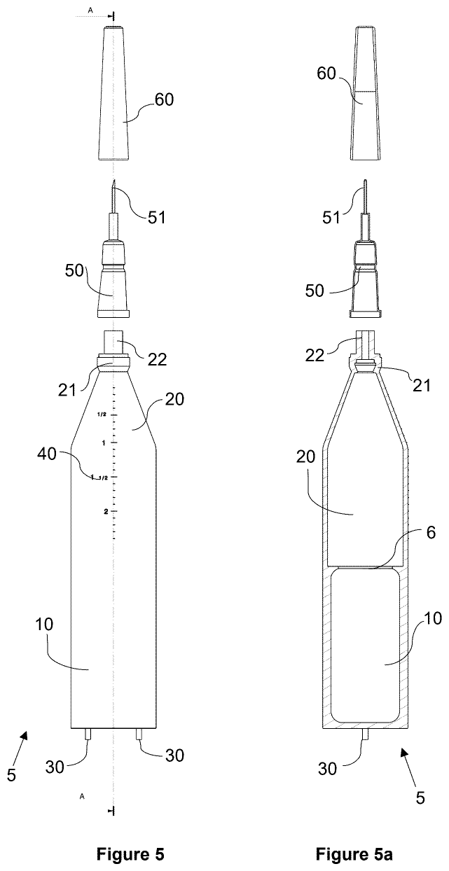

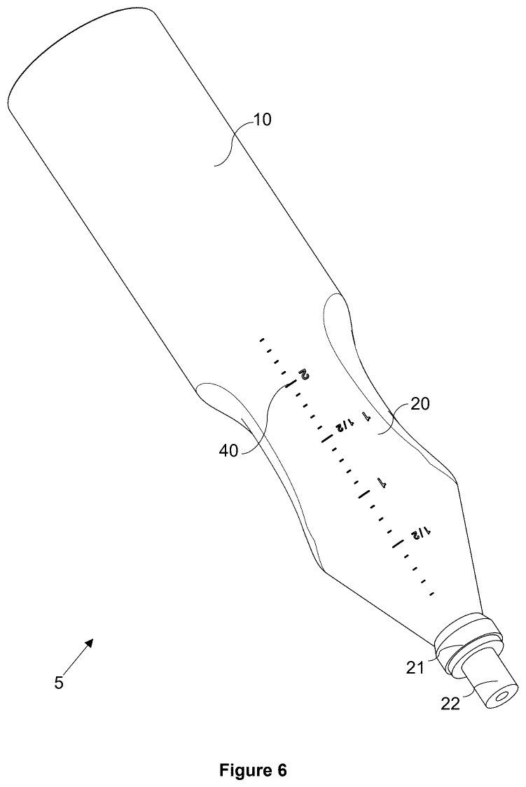

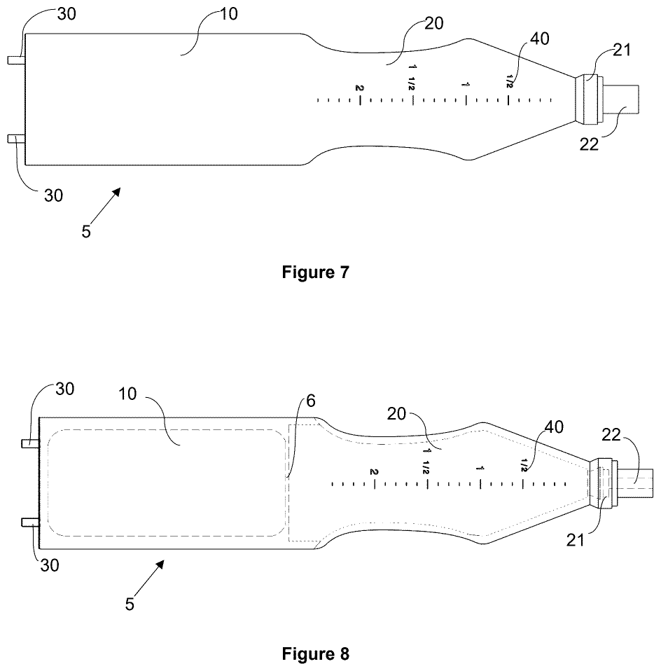

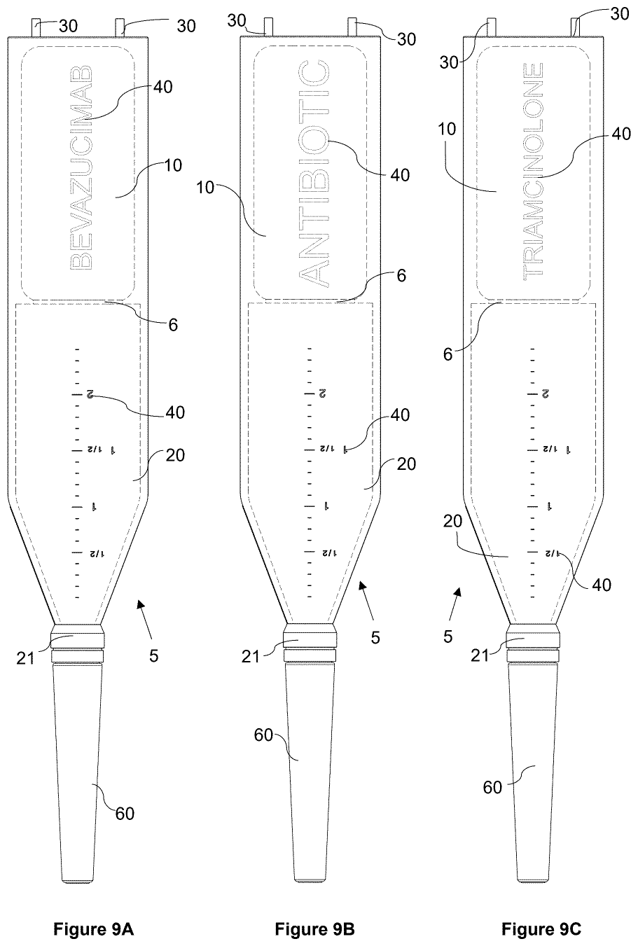

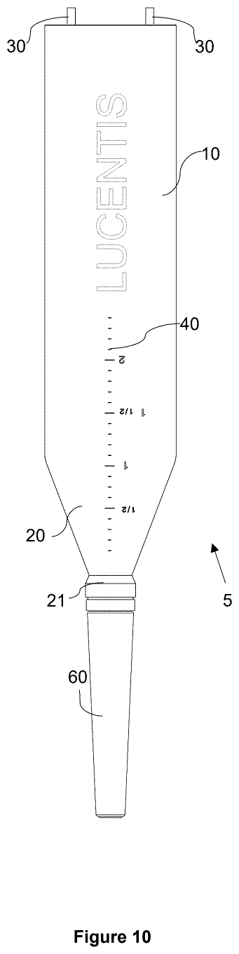

### DETAILED DESCRIPTION OF THE INVENTION

The present invention refers to a syringe for intraocular injection formed by a syringe structure free of plunger or piston with a compressible configuration in a portion thereof that allows depositing a load of a solution in the eye of a patient.

This innovative injector device was designed to simplify, facilitate, decrease operator error, and reduce adverse outcomes by providing a more ergonomic technique for intraocular injections.

This is a small disposable closed, collapsible portion, without a rigid tube and piston, especially designed for intravitreal injections through a small needle.

In one aspect of the invention, it provides a breakable seal where a smaller needle is connected to a compressible part of the syringe and after penetrating the scleral wall with the needle the drug is slowly released by compressing a compressible area of the syringe with the thumb, index and middle fingers, which represent the best hand position for the function.

Therefore, a problem that must be solved by a first aspect of the present invention is to provide a syringe for intraocular injection that does not have a plunger or piston within its structure that requires to be moved during an intraocular injection procedure.

This problem is solved by the subject matter of the independent claim, wherein the dependent claims refer to preferred embodiments of the first aspect of the present invention.

The syringe for intraocular injection preferably comprises a casing (5) that houses or forms a reservoir for a drug, in particular a fluid drug such as a liquid drug. The casing (5) preferably comprises an outer structure of the intraocular injection device, that is, a structure comprising an outer surface of the intraocular injection device. In particular, the casing (5) also serves as a holding means for the injection device, with which a user holds the injection device for operation where the injection device is a manually operated syringe where to proceed with an injection and discharge sequence requires the application of compression force to a portion of the casing (5).

Accordingly, according to a preferred embodiment, the casing (5) comprises a first casing part (10) and a second casing part (20) which is, in particular, different from the first casing part (10), i.e., is not a simple continuation of the first casing part (10) although the second casing part (20) is connected to the first casing part (10), in particular in such a way that the first casing part (10) and the second casing part (20) cannot move relative to each other in an axial direction of the intraocular injection device.

The axial direction of the intraocular injection syringe is a direction extending in the direction of a longitudinal axis of the injection device, for example, a cylinder axis if the injection device has a substantially cylindrical shape and during injection, a distal end of the injection device comprising a needle point in particular towards a location of the patient's body in which the injection is to be performed, that is, an injection location and particularly the eye of a patient.

The first casing part (10) is a part of the casing (5) that is located in an upper part of the syringe for intraocular injection, said upper part being understood as the part of the syringe that is furthest from the needle, while the second casing part (20) is located more towards a lower part of the syringe for intraocular injection, said lower part being understood as the part of the syringe for intraocular injection that is closest to a needle.

The first casing part (10) and the second casing part (20) can be understood as tanks for a solution where both are sealed or isolated from the outside but have a means of passage inside the syringe (6) that allows the fluid to be present in both internal parts of the casing (5), this being an integral feature of the casing (5) is present in the form of a syringe reservoir for intraocular injection and is filled with a medical fluid to be injected.

In particular, the tanks or reservoirs are formed integrally with the casing (5) and are located in both the first casing part (10) and the second casing part (20), in particular the first casing part (10) has a more rigid configuration of its delimitations than the second part of the casing (20) in order to protect the contents of the casing (5) from bumps or falls, while the second casing part (20) maintains a compressible configuration to achieve the expulsion of the fluid from the inside of the syringe towards the eye of a patient, said compressible configuration being allowed by the reduction of the thickness of the walls of the second part of the casing (20) with respect to the thickness of the walls of the first part of the casing (10) preferably said reduction being at least two orders smaller.

During the operation of the intraocular injection device, in particular when there is an injection event in an eye of a patient, the user or operator of the syringe presses with the index fingers and thumb or with the index thumb and middle fingers the second casing part (20) for depositing the prefilled medication in the patient's eye by compressing the walls the second part of the casing (20) in view of the fact that the pressure of the medical fluid housed inside the casing is inversely proportional to the volume occupied by compressing the walls of the second part casing part (20) the volume of the medical fluid inside it is reduced, which increases the pressure exerted on it and in this case, the pressure exerted on the medical fluid is greater than the external atmospheric pressure, causing it to be expelled from the casing through its breakable opening. When pressure is no longer exerted on the walls of the casing, the volume of the fluid expands again and the pressure decreases, allowing fluid expulsion to cease.

The movement of medical fluid is also affected by the viscosity of the fluid, which is its resistance to flow. By compressing the walls of the container and increasing the pressure, the viscosity of the medical fluid is reduced, making it easier to exit the casing.

According to a further aspect of the invention, the casing (5) has reference means (30) in the upper part or at the opposite end of the end where the syringe is connected, where these comprise at least a pair of extrusions, preferably of quadrangular section, which are arranged symmetrically with respect to the upper part of the upper part of the casing and are used as a reference for defining a radius of injection application in the eye of a patient. Preferably the separation between these reference media extrusions (30) is four millimeters.

According to an additional aspect of the invention, the casing (5) has means for identifying the dose amount or graduation (40) as a reference during the application of a dose as well as for signaling the type of drug contained inside the casing (5).

According to a further aspect of the invention, the casing (5) has a breakable needle base connector (51), which is arranged at the end of the casing closest to the connection with the syringe and where it preferably has an extension (21) that functions as a stop for the base of the needle as well as an extension (22) that is centrally coincident with the extension where the needle base connector (51) can be attached by means of pressure due to the difference in diameters. An alternative embodiment comprises that the needle connector means has a threaded rib for coupling the base of the needle.

The needle (50) of the syringe for intraocular injection that is the object of the present invention has a hollow metal body on its internal part that has a beveled end where said metal body is coupled to a needle base (51) that is coupled with the half needle connector.

An alternative embodiment comprises a needle cover (60) that protects the needle from impacts or movements that could damage it. This needle cover is held on from the needle base and is removable.

The casing of the syringe for intraocular injection as well as the reference means (30) can be made of a polymeric material, particularly polypropylene or polyethylene, making it light, economical and easy to dispose of after use and are preferably manufactured by a process plastic injection, which consists of melting the plastic into pellets and then injecting it into a mold of the desired shape, in this case the shape of the casing, and once the plastic cools and solidifies in the mold, it is removed and proceed to cut any excess material. Optionally, the tip (where the needle is located) can be assembled before sterilizing and packaging the syringe, as well as subjecting it to a scale or graduation stamping procedure on the casing that indicates the volume of liquid being injected, and can be subjected to a screen-printing process, applying ink with a graduated mold and then letting it dry. Once the scale has been applied, each syringe must be checked and calibrated to ensure accuracy before use. Other syringe grading procedures consist of manufacturing a mold with graduation included where said mold already has the graduation and when the plastic is injected it will take the shape of the graduates due to the action of pressure and temperature in the mold.

The syringe for intraocular injection can be transparent or non-transparent, since for the syringe to be transparent, the plastic must go through a bubble polymerization process, which is based on making a kind of foam and which is full of bubbles, this It gives the plastic a touch of transparency and it is not necessary to use chemicals at this stage of the process since these can leave residues that could be harmful to health, while for a syringe to be non-transparent, plastic is simply used that cannot pass through the polymerization process and therefore does not acquire the transparency that chemicals give in the last stage of manufacturing.

Regarding the needle, it is preferably made of a metallic material such as stainless steel and can be type 27G, 30G, or 32G, depending on the diameter size and can also be made of plastic or polypropylene and have a sharp tip and a conical shape to facilitate entry into the skin and allow the injection of the liquid in a controlled manner. Additionally, the needle has a hollow part, called the lumen, through which the liquid from the syringe passes. In the case of the present invention, the needle for eye injection has an exceptionally fine lumen and can vary in length depending on the type of injection to be performed. For example, for intravitreal injections, the lumen may be longer to allow the medication to reach the back of the eye. In addition to size and material, needles can also have different additional features, such as a silicone coating on the surface that makes insertion easier and reduces friction when retracted from the eye. They may also have a locking system to prevent contamination or a retractable tip for added security. The lengths normally used are 12.7 mm, 25 mm and 38 mm and the diameter can vary between 0.30 mm and 0.70 mm.

Example of Surgical Technique with the Syringe for Intraocular Injection in a Single Intravitreal Injection:
I. Injection volume: An injection volume of 0.05 ml is the most used. The maximum safe volume to inject without preinjection paracentesis is believed to be 0.1 mL to 0.2 mL.
II. Needle selection: Needle size varies depending on the substance injected; 27 G needles are often used for crystalline substances such as triamcinolone acetonide and 30 G needles are commonly used for the anti-VEGF agents ranibizumab, bevacizumab, and aflibercept. Smaller, shaping needles require less force to penetrate and cause less drug reflux. Since a smaller needle size can decrease patient discomfort.

Place the patient in an examination chair reclined at 30 degrees.

Verify the correct patient, correct eye, and medication. If bilateral injections are to be performed, ensure that the batch numbers of the medication are different, that is, different compositions.

Set up a sterile field on the tray table.

Place 1 drop of proparacaine in the eye to be injected.

Anesthetize: Take two cotton swabs soaked in 4% lidocaine and hold them over the injection site for 60 seconds, repeat two more times.

All medical staff in the room must wear a mask and the patient is asked not to speak during the procedure.

Put on sterile gloves.

Insert the eyelid speculum.

Apply one drop of Betadine 5% to the injection site and wait 30 seconds.

Mark a supertemporal injection site 3.5 to 4 mm from the limbus using the reference media (30).

Apply a final drop of Betadine 5% to the previously marked injection site, followed immediately by:

After inserting the small needle (30G-030×4 mm) (depending on medications) obliquely through the scleral wall, the drug is slowly released by the progressive and well-controlled flattening of the intraocular injection syringe between the thumb, the index, and the middle finger.

Once the injection is performed, the pressure is maintained until the intraocular injection syringe is removed from the eye, to prevent reflux of the drug into the device. Immediately cover the wound with a cotton-tipped applicator for 5 seconds.

Remove the speculum from the eyelid.

Verify the absence of pulsation of the central retinal artery.

While the present disclosure has been described with reference to various embodiments, it will be understood that these embodiments are illustrative and that the scope of the disclosure is not limited to them, and variations, modifications, additions, and improvements are possible. More generally, embodiments in accordance with the present disclosure have been described in the context or particular embodiments. Functionality may be separated or combined in blocks differently in various embodiments of the disclosure or described with different terminology. These and other variations, modifications, additions, and improvements may fall within the scope of the disclosure as defined in the claims that follow.

1. A syringe for intraocular injection comprising:
a sealed and externally isolated casing (5) that houses a liquid drug, wherein the casing (5) comprises a first casing part (10) joined to a second casing part (20) where both are tanks for the liquid drug and comprise an intermediate central passage device (6) that allows the liquid drug to be present in both internal parts of the casing (5),
wherein the first casing part (10) has a configuration more rigid than the second part of the casing (20), wherein the second part of the casing maintains a compressible configuration as well as a breakable termination where the base (51) of a needle (50) is connected to achieve the expulsion of the liquid drug, said configuration being compressible allowed by the reduction of the thickness of the walls of the second part of the casing (20) with respect to the thickness of the walls of the first part of the casing (10).

2. The syringe for intraocular injection according to claim 1, wherein the casing (5) has reference device (30) at the top or at the opposite end of the end where the needle is connected.

3. The syringe for intraocular injection according to claim 2, wherein the reference device (30) is a pair of extrusions of quadrangular section arranged symmetrically with respect to the upper part of the casing.

4. The syringe for intraocular injection according to claim 2, wherein the reference device (30) has a separation of four millimeters.

5. The syringe for intraocular injection according to claim 2, wherein the walls of the second part of the casing (20) are two orders smaller in thickness with respect to the thickness of the walls of the first part of the casing (10).

6. The syringe for intraocular injection according to claim 1, wherein the casing has an identifying device for identifying the dose amount, graduation (40) or content.

7. The syringe for intraocular injection according to claim 1, wherein the breakable end for needle connection has an extension (21) and an extension (22) centrally coincident with the extension (21).

8. The syringe for intraocular injection according to claim 1, wherein the breakable end comprises a needle connector means with a threaded rib for coupling the base (51) of the needle (50).

9. The syringe for intraocular injection according to claim 1, wherein the needle (50) has a hollow metal body in its internal part that has a beveled end.

10. The syringe for intraocular injection according to claim 1, wherein the needle (50) has a removable needle cover (60).

11. The syringe for intraocular injection according to claim 1, wherein the casing and the reference device (30) are made of a polymeric material such as polypropylene or polyethylene.

12. The syringe for intraocular injection according to claim 1, wherein the syringe is transparent or non-transparent.

13. The syringe for intraocular injection according to claim 1, wherein the needle (50) is of type 27G, 30G, or 32G.

14. The syringe for intraocular injection according to claim 1, wherein the needle (50) has a locking system.

15. The syringe for intraocular injection according to claim 1, wherein that the needle (50) has a length of 12.7 mm, 25 mm or 38 mm and a diameter of between 0.30 mm and 0.70 mm.
